(19) United States
(12) Patent Application Publication
(10) Pub. No.: US 2005/0013805 A1
(43) Pub. Date: Jan. 20, 2005

(54) Device and method for delivery of rapidly separating body of fluid, forming bone reconstruction medium

(76) Inventor: Itzhak Tavori, Herzelia (IL)

Correspondence Address: DR. MARK FRIEDMAN LTD.;c/o Bill Polkinghorn, Discovery Dispatch, 9003 Florin Way, Upper Marlboro, MD, 20772, US

(21) Appl. No.: 10/890,169

(22) Filed: Jul. 14, 2004

(30) Foreign Application Priority Data
Jul. 15, 2003 (IL) 156945

(51) Int. Cl. A61K 45/00; A61M 31/00

(52) U.S. Cl. 424/93.7; 604/500

(57) ABSTRACT

Delivery assembly for a liquid solution and granulated flakes of bone material. The device is capable of delivering multi component compound such as trinaric system, a liquid solution, living tissue cells and granulated flakes of bone material, or multi compound, to form a bone reconstruction compound and delivering the same. The delivered body of fluid is subjected to static head and dynamic head unclogging settled components and hold them as a body of fluid. The device includes a reservoir, a delivery tube, a moving member, designed to move and provide force or motion vector to the body of fluid in said tube, said reservoir is connected to said delivery tube with a passage allowing body of fluid to pass either by gravity, dynamic head provided by mixing device, or by static head.

### FIELD OF THE INVENTION

The present invention relates generally to a delivery assembly, one that is particularly suited for delivering rapidly separated body of fluid composed of a liquid solution, living tissue cells and granulated flakes of bone, or simulated bone material to form a bone reconstruction medium and delivering a bone reconstruction medium with a minimal invasive surgery.

### BACKGROUND OF THE INVENTION

In many surgical procedures, particularly orthopedic procedures, it is common practice to deliver body of fluid composed of liquid part and solid fraction. Most common practice is to deliver bone cement. In special cases, a trinaric body of fluid, composed of a liquid solution, living tissue cells and granulated flakes of bone material is required to be delivered. By a non-limiting example, in a femur bone open major surgery, required to remove bone parts, or bone marrow, the healing process is relatively long and traumatic to the patient. It is required to deliver the body of fluid with minimally invasive procedure. When introducing a prosthetic device, enhanced growth of bone around and into the prothesis is advisable to allow minimal healing and recovery time.

Other uses of such a delivery system include but not limited to, repairing or mending bone fractures or shattered bone, during face reconstruction after major trauma, during dental surgery. Moreover, delivered compound may be used together with as a drug delivery long term release system, whereby the body of fluid includes desired drugs and applied to a specific surgical site. Another application may be used together with body temperature or UV cured foundation, used to secure and hold bone and delivered compounds in a specific surgical site. As a non-limiting example, such foundation compound may be designed to be absorbed by the body over time. Because of the necessity for a fairly quick surgical procedure, a surgical assistant almost universally prepares the delivered body of fluid during the course of the operation in the sterile operating room.

Currently, delivered components forming body of fluid are mixed in a mixing bowl and, once fully mixed, the body of fluid is manually transferred from the mixing bowl to a dispensing reservoir of a dispensing device. Typically, devices similar to large diameter injection syringe or caulking guns are employed for dispensing the fully mixed body of fluid to the desired location in the surgical site. These devices relay on static head or hydrostatic pressure, to force the body of fluid threw an orifice transferring potential energy into a kinetic one.

Such device is disclosed in U.S. Pat. No. 5,551,778 to Hauke et al, another devices are disclosed in U.S. Pat. No. 5,876,116 to Barker et al, U.S. Pat. No. 5,893,488 Hoag et al. U.S. Pat. No. 6,019,765 Thornhill et al. U.S. Pat. No. 6,045,555 Smith et al. U.S. Pat. No. 6,048,346 Reiley et al. U.S. Pat. No. 6,083,229 Constantz et al. U.S. Pat. No. 6,086,594 Brown U.S. Pat. No. 6,149,655 Constantz et al. U.S. Pat. No. 5,553,754 Dentler, do not address the basic requirements and constrains of delivered body of fluid, composed of a liquid solution, living tissue cells and granulated flakes of bone material. The body of fluid is characterized by several constrains, as compound tend to rapidly separate into its components, the solid fracture tend to plug the delivery tube, high pressure, high velocity delivery is not advisable with living tissue components, coagulation may occur, non even delivery of body of fluid may occur as solid fracture tend to separate during delivery in the delivery tube, and delivered compound may be lean in one or more of its components. The higher the shear rates, the suspension dilates and enters the grain-inertia regime, where the shear stress increases quadratically with the shear rate.

In long flow, especially in small lumen diameters, or when L/D is above 10, the shear stress increased with increasing normal force. In such a quasistatic regime, the particles interlock. The quasistatic regime is dominated by short-range frictional contacts between the particles. It occurs at low strain rates under the application of compressive stress, and the stresses show strain rate independence. Successful models for the quasistatic regime have been based on Coulomb friction interactions, which are relatively insensitive to shear rate. In the suspension literature, wall slip is usually attributed to particle migration and the formation of wall slip behavior, created in unsteady flow, which is frequently referred to as “stick-slip” behavior. Stick slip is observed at low interstitial fluid viscosities, small ratios of lumen size to particle size, low shear rates, and high suspension concentration that can cause of plug flow. The flow regime turns into quasistatic regime where the particles interlock.

### SUMMARY OF THE INVENTION

The present invention provides a body of fluid delivery assembly for a liquid solution, living tissue cells and granulated flakes of bone material to form a bone reconstruction compound and delivering the same. The delivered body of fluid is subjected to static head as gravity or hydrostatic pressure, and dynamic head designed to provide movement to compound of body of fluid, unclog settled components and hold them as a body of fluid. The device includes a reservoir, said reservoir may further include a mixing device, said reservoir may include a static head device, a delivery tube, said delivery tube includes a moving member, designed to move and provide force or motion vector to the body of fluid in said tube, said reservoir is connected to said delivery tube with a passage allowing body of fluid to pass either by gravity, dynamic head provided by mixing device, or by static head.

Accordingly, the present invention provides a method comprising the steps of providing minimal cut threw the flesh, providing access surgical site possibly by a small diameter hole in the bone to the surgical site, inserting delivery tube into surgical site, filling reservoir with body of fluid, operating the delivery system, delivering the body of fluid to surgical site, retracting delivery tube outwardly from the surgical site while maintaining delivery until out of the bone, removing device from patient.

### BRIEF DESCRIPTION OF THE DRAWINGS

FIG. 1 is describing simplified device in accordance of the first embodiment of the present invention;

FIG. 2 describing simplified physical behavior of a component of body of fluid in accordance of the first embodiment of the present invention;

FIG. 3 describing simplified device in accordance of the second embodiment of the present invention.

FIG. 4 describing simplified device in accordance of the third embodiment of the present invention.

FIG. 5 describing simplified device in accordance of the fourth embodiment of the present invention.

FIG. 6 describing simplified device with as operated.

FIG. 7 describing simplified device in accordance of the fifth embodiment of the present invention.

FIG. 8 describing is describing shapes of dynamic head provider.

FIG. 9 describing is describing delivery tube.

FIG. 10 is describing the usage of device 10.

### DETAILED DESCRIPTION

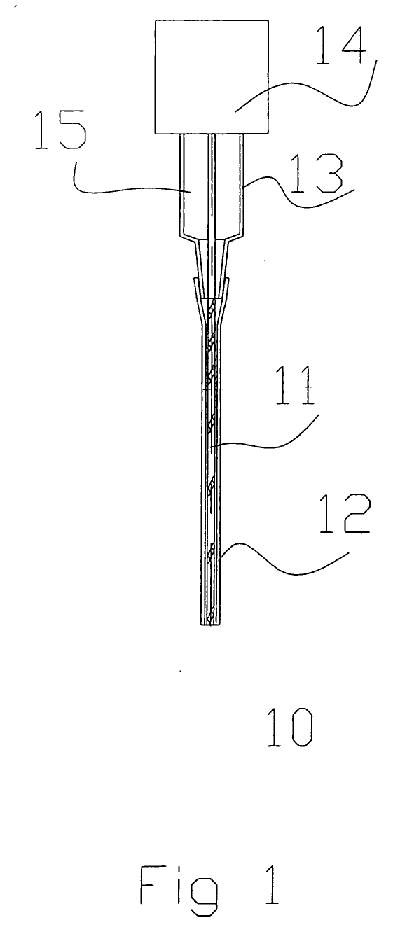

FIG. 1 is describing device 10. Device 10 having a delivery tube 12, dynamic head provider 11, reservoir 13 and power generating ability 14.

Reservoir 13 is designed to hold body of fluid 15, required to be delivered.

Additionally, Reservoir 13 is designed to provide body of fluid 15 static head or hydrostatic pressure. Static head can also be produced due to gravity. Reservoir 13 can be realized as a standard component. As a non-limiting example, a glass or flexible Erlenmeyer bottle, or with reduced volume capability, such as common syringe. Special design of reservoir 13 may include flexibility to provide static head by a simple fmger squeeze, thus reducing reservoir 13 volume. Dynamic head provider 11 is designed to have relative movement, either rotary, reciprocating or oscillating, to body of fluid 15 to pass threw the delivery tube 12. By having such movement, Dynamic head provider 11 add another component of force or motion to the flow in which body of fluid 15 is exposed to. This component is used to break settling of body of fluid 15, when passing via delivery tube 12, or present in reservoir 13. Dynamic head provider 11, may be realized integrally with delivery tube 12, or reservoir 13 thus reducing components required to realize device 10. Power generating ability 14 is used to provide agitation or other movement to hold components of body of fluid 15, dispersed and not settled. Power generating ability 14 is also used to provide relative movement between delivery tube 12, and dynamic head provider 11. Power generating ability 14, can be realized by manually tilting device 10 by hand, or by squeezing reservoir 13 by hand, or by connecting to other power source. Such a power source can be an electric, hydraulic, spring operated or pneumatic motor, capable of delivering any of rotary or reciprocating or oscillating motion. Power generating ability 14 may also be realized by subjecting delivery tube 12, or dynamic head provider 11 or complete device 10 to electromagnetic or ultrasonic fields. Numerous power source devices are present, and in operating room environment, Hall drill, available from Zimmer U.S.A, can provide rotary motion, and a Sternum saw, available from Zimmer U.S.A, or De Soutter Medical, which can provide oscillating or reciprocating motion. Moreover Power-generating ability 14 may be designed to produce oscillations between delivery tube 12, and dynamic head provider 11 due to out of phase inertial motion of the body of fluid 15, and dynamic head provider 11 system.

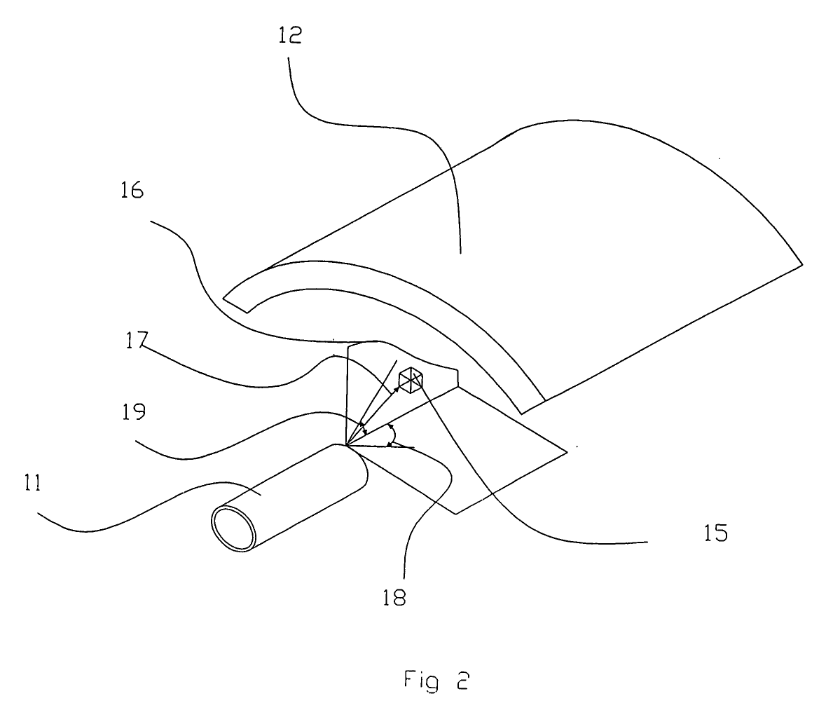

FIG. 2 is describing a part of body of fluid 15, inside device 10, and physical relationship as to forces of flow. The body of fluid 15 can not be considered as a single phase fluid, as it is composed of a liquid solution, living tissue cells and bone flakes. Moreover, the liquid solution, living tissue cells combination forms a non-Newtonian compound, with low characteristics of fluidity behavior. Such a part may be composed of either component of a body of fluid 15, and may be a liquid, gas, Living tissue cells, such as blood, stem cells, bone marrow cells and the like, Bone flacks, said bone flacks may be coated with drug compound, or with a gelling agent providing affinity to living tissue cells, or drug compound and the like, to adhere to the bone flacks, thus posing a semi solid fraction in body of fluid 15. Parts of device 10, dynamic head provider 11 and delivery tube 12 are also shown.

The shape of a part of body of fluid 15 is of irregular shape, both simple 3-D shapes such as sphere, cube and the like, and flakes as coming out of the mill, without and ordered structural shape.

A part of body of fluid 15, is subjected to a flow field, most commonly described by the following formula:

$$ρ \frac{D {V}^{->}}{D t} = ρ {B}^{->} - ∇ p + μ {∇}^{2} {V}^{->}$$

This equation is known as the Navier-Stokes equation, for Newtonian fluids. The term on the left side of the equation represents the total derivative consisting of the temporal and convective acceleration terms, of which the convective acceleration are non-linear. The terms on the right side of the equation represent the body acting forces, the pressure gradient, and the forces due to the viscosity of the fluid. The body acting forces are proportional to the wetting behavior between the particles, surface and shape and liquidic part of the body of fluid. The velocity field is proportional to the pressure drop field and can be described as graph 16, where curve proximae is upstream, and curve distalia is downstream. Of coarse, graph 16 may oscillate, and create average downstream flow, intermittent flow or upstream flow. When considering a reservoir 13 and a delivery tube 12, such a body of fluid 15 poses higher delivery phenomena. It is known from prior art that according to Bernoulli's equation, when a fluid flows steadily through a long, narrow, horizontal pipe of constant cross section, the pressure along the pipe will be constant. In practice, however, we observe a pressure drop as we move along the direction of the flow. Body of fluid 15 composed of a liquid solution, living tissue cells and bone flakes, has rapid separation characteristics, and at the far flow field, solid or semi solid particles will separate from the body of fluid 15 and will be subjected to external forces generated by dynamic head provider 11, and delivery tube 12. Moreover, flow in long constrictions with a small lumen diameter, or flow separation regions, or turbulent energy losses in cases of severe stenosis, reduce the energy content of the fluid.

When the proportion of the delivery tube 12 length and lumen diameter are above 10, the settled particles will form a plug flow, which will end as clogging delivery tube 12.

In this invention, additional vector 17 is used to bring particles from body of fluid 15, settled on delivery tube 12, back into the body of fluid 15 hence unclogging the plug formed. The vector 17 is adding another component of force or motion, to the body acting forces, coming from external source. Vector 17 is characterized by value, position, and angle of attack. The angle of attack is represented as polar coordinates 18, as θand 19 as φ. Polar coordinates are between 0 to 2 ¶ and can be subjected to a rotary or reciprocating field or any other combination regime, hence providing vector 17 to apply force or motion to body of fluid 15. Although vector 17 is shown coming from dynamic head provider 11 it also may be coming from delivery tube 12, as a result of electromagnetic or ultrasonic field, thermal energy or impact between particles. Forces generated from slip stick phenomena, from delivery tube 12, or from particles interaction are not shown. Vector 17 is adding components to the basic Navier-Stokes equation, results a modified Navier-Stokes equation:

$$ρ \frac{D {V}^{->}}{D t} = ρ {B}^{->} - ∇ p + μ {∇}^{2} {V}^{->} + {F}^{->} δ ( x , y , z , φ , θ , t , S )$$

Where the added component is dependant in value F, in position (x,y,z), in direction (Θ,φ), in time (t), and value S, resembling shape. The importance of S, relates to volume, surface area of from body of fluid 15, moment of inertia, gyration radii and other dynamic functions, generated by travel of particle of body of fluid 15 in medium. The term time (t) may be substitute with frequency (1/t). Of coarse, δ(x,y,z,φ,θ,t,S), may be a function, independent or dependant of any of its components.

Expanding this formula,

$$ρ \frac{\partial {V}^{->}}{\partial t} + ρ {V}^{->} \cdot ∇ {V}^{->} = - ∇ p + ρ { g}^{->} + μ {∇}^{2} {V}^{->} + {F }^{->} δ ( x , y , z , φ , θ , t , S )$$

Looking into the formula,

$$ρ \frac{\partial {V}^{->}}{\partial t}$$

is the local acceleration, ρ\vec{V}·∇\vec{V is} the convective acceleration, −∇p is the pressure force per unit volume, ρ\vec{g} is the body force per unit volume, μ∇²\vec{V} is the viscous forces per unit volume and \vec{F}δ(x,y,z,φ,θ,t,S) Is externally added component of force per unit of shape, Examination of the above equation shows that each term has units of force per unit volume, or, F/L3. Therefore, Added component \vec{F}δ(x,y,z,φ,θ,t,S) satisfy basic equation, as if we divide each term by a constant having those same units (F/L3), we would obtain a dimensionless equation. Furthermore, the viscosity and specific gravity values are also changing, when Vector 17 is not pure mechanical, activated by electromagnetic ultrasonic fields or pneumatic or hydraulic dissipation or impact, when heat is applied or generated. As a non limiting example, such a phenomena is present when dynamic head provider 11 is realized as a stream of a component of body of fluid 15, the local flow regime is a combination of regimes, creating a vector 17.

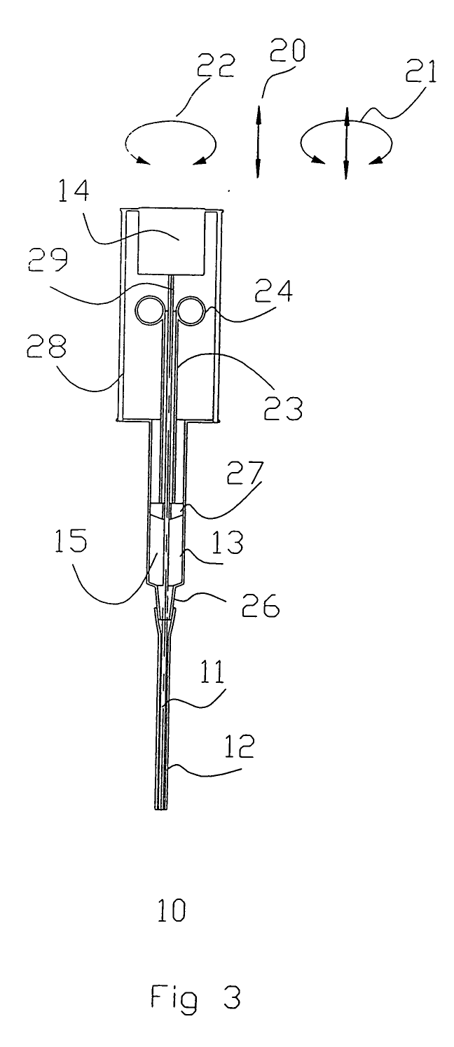

FIG. 3 is describing device 10, further including a ram 27, designed to apply pressure and push body of fluid 15 threw fimnel 26 via delivery tube 12. Funnel 26, may be angled or parallel, constitutes continuous integral part of reservoir 3. Ram 27 may be realized as standard syringe ram, or any other piston arrangement. Pusher 23 and handle 24 activate Ram 27 in reservoir 13. Pusher 23 and handle 24 are drawn schematically as can be realized as hand operated equipment, power operated include leverage as caulk guns, and equipped with metering or motorized capability. Pusher 23 may include a passage between power generating ability 14, and dynamic head provider 11. Power generating ability 14, is connected to dynamic head provider 11 via a coupling 29, capable of delivering power generated, such as rotary movement, 22, linear movement 20, or combination movement 21. Rotary movement, 22 may be either continuous or reciprocating, vibration or frequency oscillating. Linear movement 20 may be either continuous or reciprocating, vibration or frequency oscillating, either small short stroke, or full length stroke, as the length of delivery tube 12, allowing small portions of body of fluid 15 to be rammed all the way out of delivery tube 12, Combination of movement 21, both rotary and linear, in required operating regime, as independently operated, or timed linear and than timed rotary, capable of rotating and than pushing. Coupling 29 is further including capability to couple electromagnetic, ultrasonic fields, or pneumatic or hydraulic dissipation or impact. Frame 28 is described schematically, and designed to hold together components of device 10, however frame 28 may be designed integrally, thus frame 28 is imbedded into device 10.

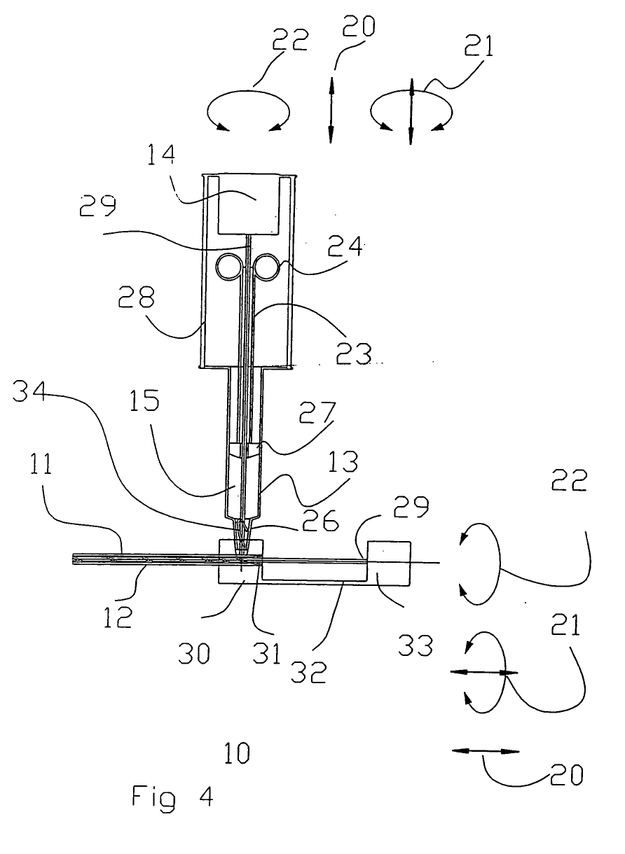

FIG. 4 is describing device 10 in an angle position. In this arrangement, hydraulic Tee joint, 30 is mounted over funnel 26, and delivery tube 12 dynamic head provider 11. Reservoir 13 is further including a mixer 34, integrated inside funnel 26, and connected to power generating ability 14 via passage in pusher 23 and coupling 29. Mixer 34 may be designed for minimum or maximum free volume inside funnel 26. moreover, mixer 34 may be designed as a syringe and a needle system, operating as to motion 20. creating oscillating motion in body of fluid 15. In such an arrangement, delivery tube 12 and dynamic head provider 11 is in an angle to reservoir 13, handle 24, pusher 23, ram 27 frame 28 coupling 29 and power generating ability 14. Hydraulic Tee joint 30, also including a seal 31, capable of transferring movement generated by power generating ability 33, and sealing body of fluid 15 from dripping proximally, outside of the distal surgical site. This angled arrangement allows linear movement 20, rotary movement, 22, combination of movement 21, to be separately introduced to dynamic head provider 11, and body of fluid 15 or other components. Frame 32, connecting hydraulic Tee joint, 30, and power generating ability 33, is described schematically, however frame 33 may be designed integrally, thus frame 33 is imbedded into device 10.

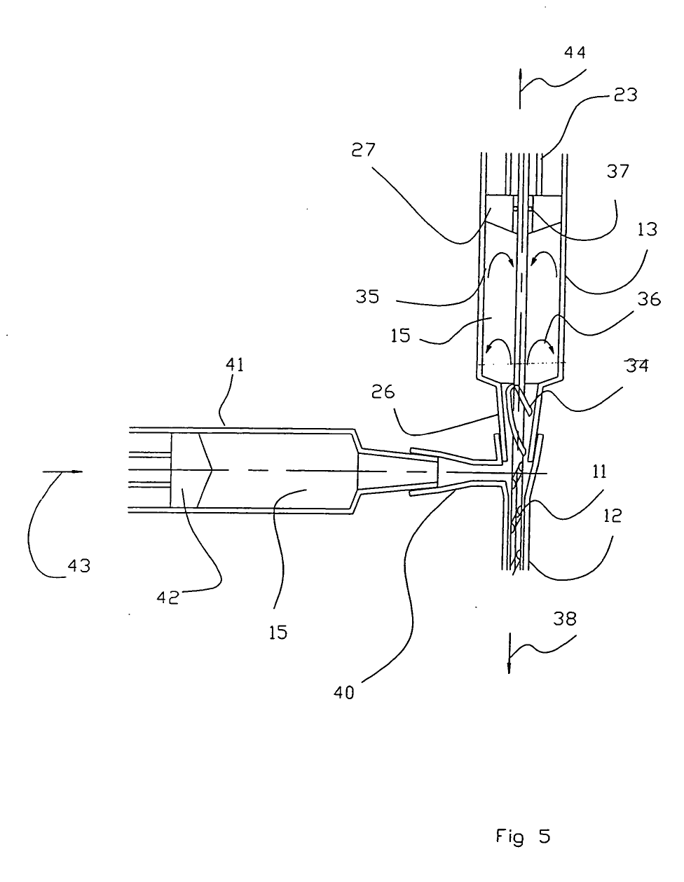

FIG. 5 is describing part of device 10 having a delivery tube 12, dynamic head provider 11, reservoir 13 and power generating ability 14. Mixer 34, connected to coupling 29 via pusher 23 inside funnel 26, can generate opposite direction flow regime 36, further mixing components of body of fluid 15, or in-direction flow regime 35, capable of dynamically powering body of fluid 15 into fimel 26 and to delivery tube 12 as to required direction 38. Moreover, Mixer 34 and dynamic head provider 11, can be realized integrally as a secondary syringe and a needle system, capable of being integrated inside ram 27 or pusher 23, creating additional flow regime when subjected to movement 20. Ram 27 further including seal 37 allowing passage of movements 20,21,22 (not shown) and sealing body of fluid 15 inside reservoir 13 and ram 27 system. Mixer 34 may be extended into reservoir 13, and can be made of flexible material such as flexible plastic or Nitinol, capable of collapsing into small volume, at the bottom of reservoir 13 or funnel 26.

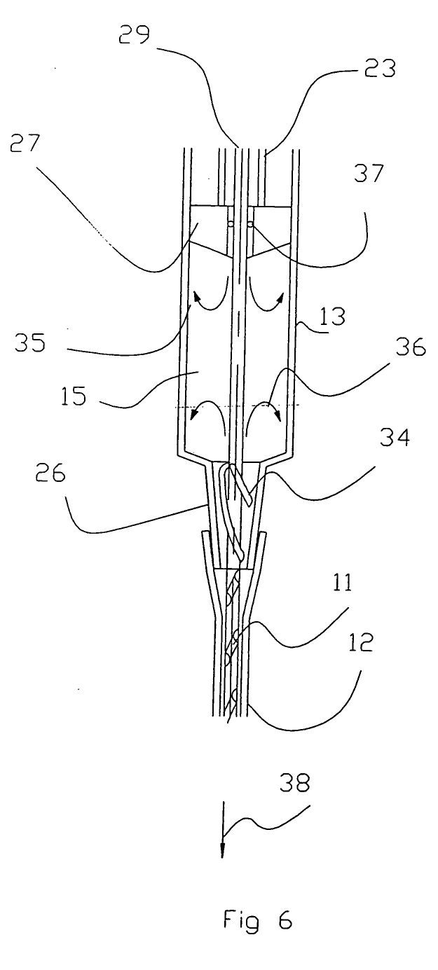

FIG. 6 is describing part of device 10, having ram 27 driven by pusher 23, minimizing reservoir 13 volume. Ram 27 includes seal 37, allowing passage of movements 20,21,22 (not shown) and sealing body of fluid 15 inside reservoir 13 and ram 27 system, via coupling 29. Inside coupling 29, seal 39 is holding, allowing passage of movements 20,21,22 (not shown) and sealing body of fluid 15 inside reservoir 13 and ram 27 system. This arrangement allow passage of independent movements 20,21,22, (not shown) between power generating ability 14, dynamic head provider 11, and mixer 34. As a non-limiting example, rotary movement can be transferred to mixer 34, and mechanical, oscillating electromagnetic or US field to dynamic head provider 11. Movements can be pure mechanical, activated by electromagnetic ultrasonic fields or pneumatic or hydraulic dissipation or impact, and can be achieved via seal 39.

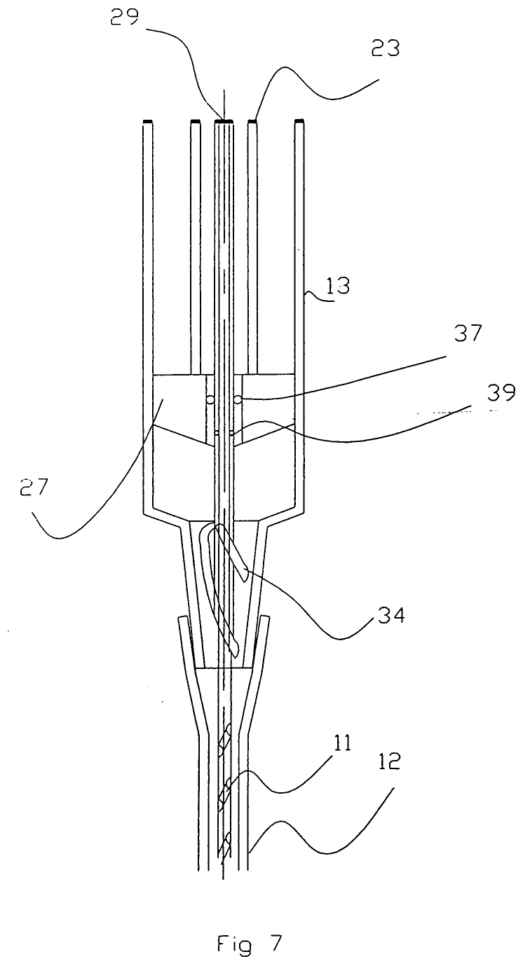

FIG. 7 is describing part of device 10, having a delivery tube 12, dynamic head provider 11, reservoir 13 and power generating ability 14. Mixer 34, connected to coupling 29 via pusher 23 inside funnel 26. Hydraulic Tee joint, 40, is mounted over funnel 26, further including possibility to connect at least a single additional reservoir 41, capable of holding body of fluid 15. When ram 42 is pushed in direction 43 and ram 27 is pulled in direction 52, body of fluid 15 is transferred from reservoir 41 to reservoir 13, providing easy loading tool and procedure. In this procedure, delivery tube 12 is temporarily plugged by an external device (not shown), or mixer 34 may be operated to draw body of fluid 15, into reservoir 13. The system of reservoir 41 and reservoir 13, allow added hydraulic movement regime, as ram 42 and ram 27 may be operated respectively, with directions 43, 52, and against directions 43, 52. Ram 42 and ram 27 are subjected to power, generated either by hand or other device, allowing reciprocating movement of body of fluid 15 in such a system. Moreover, part of body of fluid 15 may be in reservoir 13 and another part in reservoir 41, allowing “mixing when delivered” capability. Reservoir 13, with installed components, may substitute additional reservoir 41. Reservoirs 13,41 may be incorporated with metering capability, showing amount delivered. Hydraulic Tee joint, 40, may be provided with capability to hold plurality of reservoir 41, and stopcocks, (not shown), allowing removal of reservoir 41, when not required.

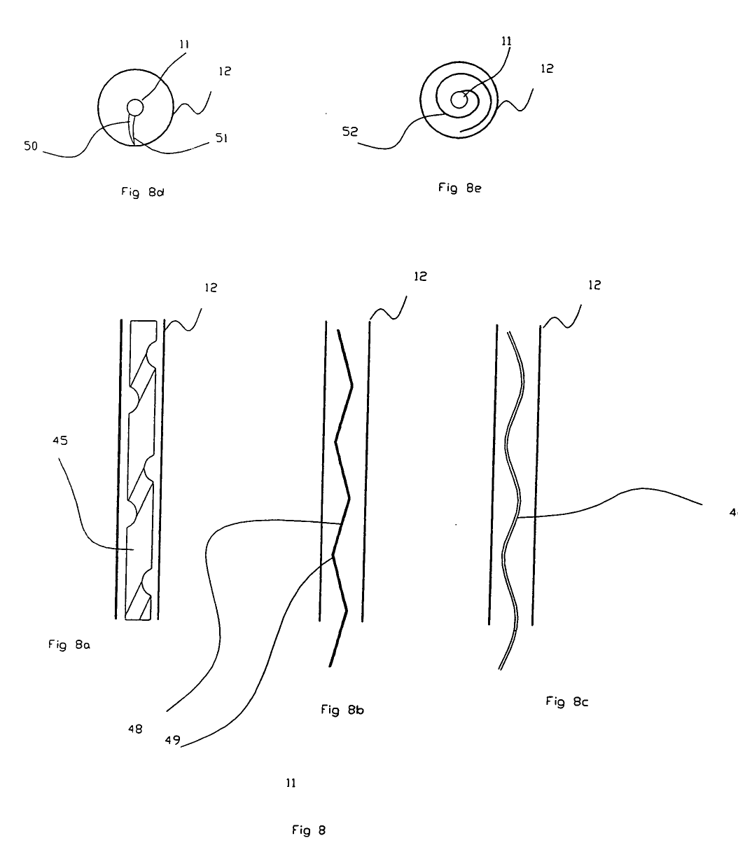

FIG. 8 is describing shapes of dynamic head provider 11. Dynamic head provider 11, may be in a shape of a rod, a cylinder, or any other shape. It may constitute a part of other components of device 10 (not shown), it may be made of relatively stiff material, such as stainless steel, or flexible material such as polymer or Nitinol, capable of rotating, oscillating or moving along a curved axis line, or partly curved axis line. Distal tip of dynamic head provider 11 is designed not to harm the tissue at surgical site. Distal tip of dynamic head provider 11, may be enclosed inside delivery tube 12, may be of soft material, when protrude distally from delivery tube 12, maybe round in shape. All arrangements of dynamic head provider 11 may be hollow, allowing passage of body of fluid 15 or a component of body of fluid 15. In FIG. 8a, Dynamic head provider 11 may be realized as a lead screw, implementing Archimedes screw principal 45. When rotated, inside delivery tube 12, body of fluid 15 (not shown), is transferred along the longitudinal axis. This arrangement is capable of delivering high pressure. The lead angle may be constant or a progressing type, capable of accelerating body of fluid 15 (not shown), when rotated at constant speed. In FIG. 8b, another arrangement is present as taking only a thread from a lead screw, 46, much like a common coil spring. This arrangement is designed to further give movement to body of fluid 15 (not shown), and breaking the plug flow. The lead angle may be of a progressing type, capable of accelerating body of fluid 15 (not shown), when rotated at constant speed. This arrangement allows minimal dimensions of the dynamic head provider 11, and delivery tube 12 system.

In FIG. 8c, another arrangement is present in collapsible form 47, composed of linear segments 48 and collapsible joints 49. When subjected to linear movement 20 (not shown) it collapses along the longitudinal axis. Balancing forces and stress in collapsible joint 49, yields spontaneous release of stress, and amplified movement. This arrangement allows ramming of body of fluid 15 by dynamic head provider 11 out of delivery tube 12. Arrangement 45,46,47, may be hollow inside, allowing additional seepage of another component, either pneumatic or hydraulic, of body of fluid 15 (not shown).

In FIG. 8d, a side view of dynamic head provider 11 is shown. The shape of a blade is holding inside delivery tube 12. Such an arrangement can be realized, as a non-limiting example, as a shape of a common spiral drill bit. Front edge 50, and trailing edge 51, constitutes a blade, which may be of various shapes. Of coarse, plurality of front edge 50, and trailing edge 51, may be used. Front edge 50, and trailing edge 51, may touch delivery tube 12, or be distant, allowing backwash flow, hence providing additional flow regime, breaking the plug flow, holding components inside body of fluid 15.

In FIG. 8e, a side view of dynamic head provider 11 is shown. In this arrangement, a dynamic head provider 11 is in a form of rotating spiral 52. Such a rotating spiral 52, when collapsing in the longitudinal axis, provides additional movement to body of fluid 15 components.

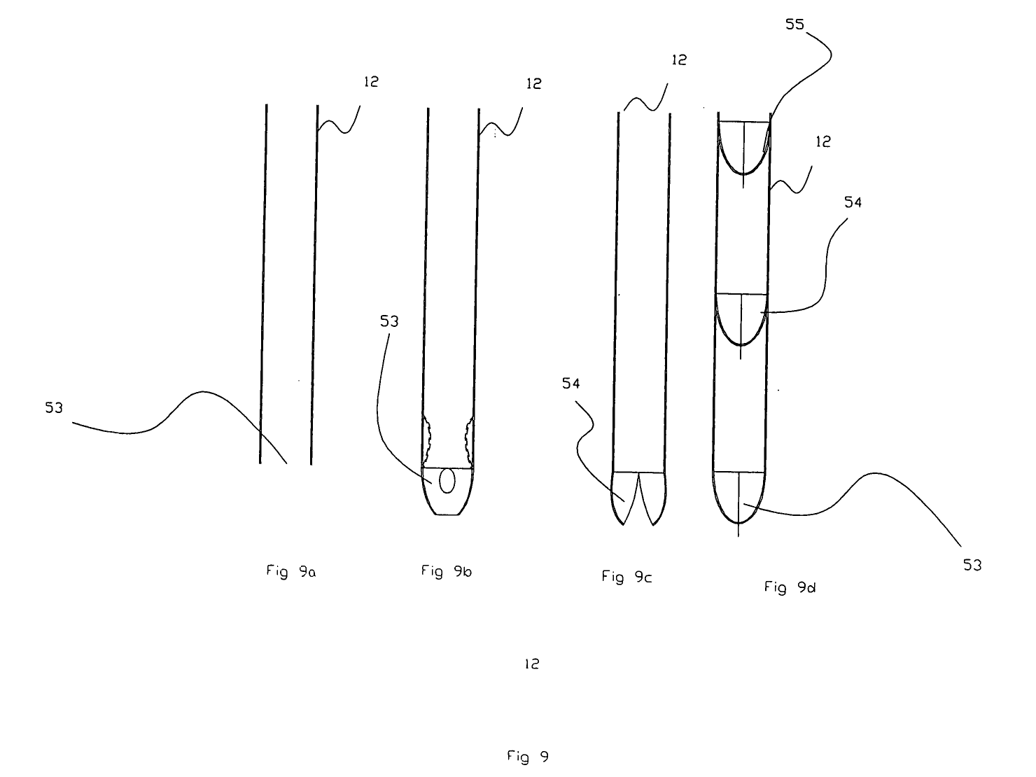

FIG. 9 is describing delivery tube 12. Delivery tube 12 can be realized as intra-vascular catheter, available from Cordis, U.S.A, a venous cannula, available from Medtronic, U.S.A., A simple tube, made out of any body compatible material of construction, either metal or polymer, of relatively stiff material, such as stainless steel, or flexible material such as polymer or Nitinol, capable of rotating, oscillating or moving along a curved axis line, or partly curved axis line.

FIG. 9a, describe the distal tip 53 of delivery tube 12, designed to allow easy insertion of delivery tube 12 threw the flesh and into surgical site. Distal tip 53 of delivery tube 12 is designed not to harm the tissue at surgical site. Distal tip 53 of delivery tube 12, may be flat, in any angle relative to longitudinal axis of delivery tube 12.

FIG. 9b further describing the distal tip 53 of delivery tube 12. Distal tip 53 of delivery tube 12, may be round or oval in shape, including orifice or plurality of orifice, much like present in venous cannula, available from Medtronic, allowing delivery of body of fluid 15, and insertion over the pin used in Intra medullary set. Distal tip 53 of delivery tube 12 may be of soft material, easily collapsible inside delivery tube 12, (shown with dashed line) when forced by external force (not shown).

FIG. 9c further describing the distal tip 53 of delivery tube 12. In this arrangement, several cuts are made to distal tip 53, to form a one-way valve 54 capable of releasing body of fluid 15 coming from inside delivery tube 12, into surgical site. When pressure at surgical site is higher than pressure generated by dynamic head provider 11 and ram 27 system, one way valve 54 is closing, holding delivered body of fluid 15 at surgical site.

FIG. 9d further introducing one-way valve 55, inside delivery tube 12. In this arrangement, reciprocating movement of body of fluid 15, is governed by one way valves 54,55, creating “peristaltic” distal advance of body of fluid 15 in portions, resulting intermittent movement. Of coarse, plurality of one-way valve 55 may be incorporated inside delivery tube 12.

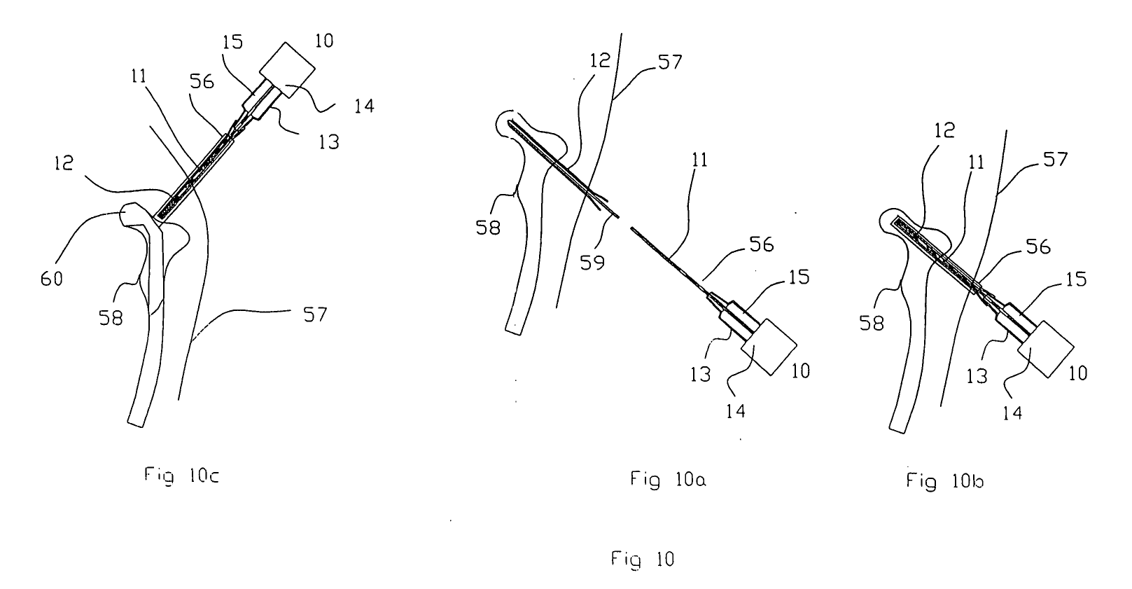

FIG. 10 is describing the usage of device 10. The following paragraph is describing usage of device 10 by a non-limiting example relating to Femur bone orthopedic surgery. Of coarse, other procedures may be performed. Described tools are included as Intra medullary set, for orthopedic surgery.

A small cut is made threw the skin and the Tensor fasciae latae muscle, all the way until the bone line 57 is schematically representing the contour of skin and flesh. The bone is prepared for puncturing. Using Hall drill, or other device, the bone 58, is drilled with small diameter drill bit, larger than delivery tube 12. Described procedures may be performed under any available imaging system, Radio, US, CT and the like. Delivery tube 12, and or dynamic head provider 11, may be integrated with radio opaque capability, allowing procedure under fluoroscopy. Materials of construction of device 10 can be made of any material suitable for use as surgical device. These materials including, but not limited to, metals, titanium, Nitinol Stainless steel, and polymer materials as pebax, PU. PE, PP, PET, PTFE PVC, or any other body compatible material. Providing delivery tube 12, and or dynamic head provider 11 of flexible material, allow delivery tube 12, and or dynamic head provider 11 to bend over and follow channel provided in the bone, as it may be in an arc shape, and the Intra medullary set, does not necessarily providing straight, round, parallel hole.

FIG. 10a, is showing status when the drill is removed leaving guiding device 59, usually in a form of a pin, distally at the surgical site, proximal end protruding out from the flesh 57. Delivery tube 12, is disassembled from device 10, and inserted over the guiding device 59. Body of fluid 15, is loaded into reservoir 13. Guiding device 59 is removed from delivery tube 12, and device 10 is assembled with Delivery tube 12, and dynamic head provider 11. Power generating ability 14, is activated and body of fluid 15 is passing into surgical site, either by gravity and dynamic head or by other methods of static head and dynamic head. Device 10 is moved distally and proximally, to provide cavity for body of fluid 15 to accumulate. Such a movement also used to pack and fill cavity, and such movement is progressing out of the bone, coming proximally. When required volume is delivered, device 10 is removed. FIG. 10b is describing fully assembled device 10. Device 10 can be use fully assembled and loaded. In such a procedure, a cannula 56 with diameter larger than delivery tube 12 is slide over the guiding device 59. The guiding device 59 is removed and fully loaded and assembled device 10 is inserted and operated. When moved distally and proximally, to provide cavity for body of fluid 15 to accumulate, the cannula 56 is retracted together with device 10.

FIG. 10c is describing procedure for enhancing growth around a prosthesis 60. This procedure can be used in case surgical site is near the skin, or in case of bone fracture Aa cut is made to expose the surgical site and fully assembled device 10 is used to deliver Body of fluid 15.

Described procedures may be performed under any available imaging system, radio, US, CT and the like. Delivery tube 12, and or dynamic head provider 11, may be integrated with radio opaque capability, allowing procedure under fluoroscopy. Materials of construction of device 10 can be made of any material suitable for use as surgical device. These materials including, but not limited to, metals, titanium, Nitinol Stainless steel, and polymer materials as pebax, PU. PE, PP, PET, PTFE PVC, or any other body compatible material. Providing delivery tube 12, and or dynamic head provider 11 of flexible material, allow delivery tube 12, and or dynamic head provider 11 to bend over and follow channel provided in the bone, as it may be in an arc shape, and the Intra medullary set, does not necessarily providing straight, round, parallel hole.

1. Device for delivery body of fluid composed of a liquid solution, and bone flakes comprising Newtonian and Non-Newtonian body of fluid;
device further including means for delivering motion vector to body of fluid;
wherein
said motion vector is holding components of body of fluid in suspension and eliminating plug flow.

2. Device according to claim 1,
wherein
said body of fluid further includes bone marrow.

3. Device according to claim 1,
wherein
said body of fluid further includes stem cells.

4. Device according to claim 1, Said bone flacks compound comprising body of fluid further includes coating with gelling agent,
wherein
said bone flacks gelling agent is holding live tissue cells coating over bone flacks.

5. Device according to claim 1,
wherein
said body of fluid further includes long term drug delivery system.

6. Device according to claim 1, providing motion vector
wherein
said motion vector is described in modified Navier-Stokes equation,
$$ρ \frac{D {V}^{->}}{D t} = ρ {B}^{->} - ∇ p + μ {∇}^{2} {V}^{->} + {F}^{->} δ ( x , y , z , φ , θ , t , S ) .$$

7. Device according to claim 1,
wherein
materials of construction are selected any of a group:
titanium,
Nitinol,
stainless steel,
pebax,
PU. PE, PP,
PET,
PTFE.

8. Device for delivery body of fluid composed of a liquid solution, living tissue cells and bone flakes;
device including reservoir for holding body of fluid, said device further including provision for static head, provision for dynamic head, provisions for power generating ability, and a delivery tube,
and
means for delivering motion vector to body of fluid;
wherein
applying static head makes body of fluid run inside delivery tube
and
means for delivering motion vector to body of fluid is braking the
plug flow, holding components of body of fluid in suspension flow.

9. Device as to claim 8
wherein
static head produced due to gravity.

10. Device as to claim 8
wherein
means for delivering motion vector to body of fluid is selected any of a group:
mechanically moving member,
ultrasound oscillations,
electromagnetic fields,
hydraulic bleeding,
hydraulic impact,
pneumatic seepage.

11. Device as to claim 8
wherein
delivering motion vector to body of fluid is selected any of a group:
Archimedes lead screw principle, with or without provision for backwash,
a thread, in a form of a coil spring
a thread, in a form of a spiral,
a ram, capable of ramming small portions of body of fluid all the way out of delivery tube,
a collapsible ram,
hollow member,
integrally with delivery tube,
integrally with reservoir,
integrally with device.

12. Device as to claim 8, further including a mixer.

13. Device as to claim 8, including power generating ability
wherein
movements are any selected from a group:
linear movement,
rotary movement,
linear and rotary movement,
timed linear and then timed rotary,
thus producing required flow regime for body of fluid, braking the plug flow, holding components of body of fluid in suspension flow.

14. Device according to claim 8, further include pusher, couplings and seals
wherein
coupling and seals may extend via pusher, or may be in an angle to each other, providing capability of transferring movements either separately or together, while seals keep body of fluid inside the device.

15. Device according to claim 8,
wherein
delivery tube includes at least a single one way valve.

16. Device as to claim 8, further including plurality of reservoir.

17. Device as to claim 16,
wherein
any of plurality reservoir is used to maintain flow of body of fluid between reservoirs,
delivering motion vector to body of fluid, braking the plug flow, holding components of body of fluid in suspension flow.

18. Device as to claim 16,
wherein
any of plurality reservoir is used to add another component to body of fluid,
providing mixing when delivered capability.

19. Device for delivery body of fluid composed of a liquid solution, living tissue cells and bone flakes;
device including reservoir for holding body of fluid, said reservoir further including provision for static head, provision for dynamic head,
provisions for power generating ability, and a delivery tube,
device is further including means for delivering motion vector to body of fluid,
said means for delivering motion vector to body of fluid is braking the plug flow, holding components of body of fluid in suspension flow,
wherein
said means for delivering motion vector to body of fluid, and said delivery tube are of flexible material, capable of operating when longitudinal axis is curved.

20. A method for minimally invasive surgery, said surgery providing delivery body of fluid composed of a liquid solution, living tissue cells and bone flakes susceptible of fast settling and plug flow, and, a delivery device, said device including reservoir for holding body of fluid, said device further including provision for static head, provision for dynamic head, provisions for power generating ability, and a delivery tube,
wherein
loading reservoir of said device with body of fluid compound,
providing minimal cut threw the skin and flesh,
preparing the bone for delivery of said body of fluid,
inserting delivery tube of said delivery device threw the flesh into the surgical site operating said power generating ability,
providing static head,
providing dynamic head, said dynamic head having means for delivering motion vector to body of fluid is braking the plug flow, and holding components of body of fluid in suspension flow,
delivering body of fluid to surgical site,
advancing said delivery device around surgical site,
advancing said delivery device out of surgical site while maintaining delivery, until all required amount of said body of fluid is delivered,
removing said delivery device out of the flesh and skin.

21. A method according to claim 11, further including preparing the bone for delivery of said body of fluid,
providing an opening into the bone, larger than said delivery tube,
inserting a cannula larger than said delivery tube,
inserting said delivery device threw said cannula into surgical site,
operating said power generating ability,
providing static head,
providing dynamic head, said dynamic head having means for delivering motion vector to body of fluid is braking the plug flow, and
holding components of body of fluid in suspension flow,
delivering body of fluid to surgical site advancing said delivery device around surgical site advancing said delivery device out of surgical site while maintaining delivery, until all required amount of said body of fluid is delivered,
removing said delivery device out of the flesh and skin.

22. A method according to claim 11, further including preparing the bone for delivery of said body of fluid,
providing an opening into the bone, larger than said delivery tube,
inserting a guiding device smaller than said delivery tube,
inserting said delivery tube separate from said delivery device over the guiding device into surgical site,
removing the guiding device,
assembling said deliver device over said delivery tube comprising complete delivery device,
operating said power generating ability,
providing static head,
providing dynamic head, said dynamic head having means for delivering motion,
vector to body of fluid is braking the plug flow,
holding components of body of fluid in suspension flow,
delivering body of fluid to surgical site,
advancing said delivery device around surgical site,
advancing said delivery device out of surgical site while maintaining delivery, until all required amount of said body of fluid is delivered,
removing said delivery device out of the flesh and skin.
